(19) United States
(12) Patent Application Publication
(10) Pub. No.: US 2006/0024271 A1
(43) Pub. Date: Feb. 2, 2006

(54) Treatments for viral infections using IFN cytokines and ribavirin, alone or in combination

(76) Inventors: Ken Alibek, Fairfax, VA (US); Ge Liu, Rockville, MD (US); Svetlana Hopkins, Arlington, VA (US)

Correspondence Address: HOGAN & HARTSON L.L.P., Suite 700, 2049 Century Park East, Los Angeles, CA, 90067, US

(21) Appl. No.: 11/033,828

(22) Filed: Jan. 13, 2005

Related U.S. Application Data
(60) Provisional application No. 60/536,504, filed on Jan. 15, 2004.

(51) Int. Cl. A61K 38/21; A61K 9/127; A61K 31/7056

(52) U.S. Cl. 424/85.6; 424/85.7; 514/43; 424/450

(57) ABSTRACT

A treatment or prophylaxis for viral infection using interferon (IFN) cytokines alone or in combination with ribavirin is provided. The treatments and prophylaxis allow for lowered dosages of IFNs, reduced pro-inflammatory responses, and delays the initiation time and reduced frequency of the IFN treatment required.

### REFERENCE TO CROSS-RELATED APPLICATIONS

This application claims the benefit of U.S. provisional application no. 60/536,504, filed Jan. 15, 2004, (attorney docket no. 08675-6045), which is incorporated herein by reference.

This invention was made with Government support under MDA-972-02-C-0067 and W911NF-04-C-0046 awarded by DARPA. The Government has certain rights in the invention.

### BACKGROUND OF THE INVENTION

This invention relates to treatments for viral infections.

Routine vaccinations for smallpox were discontinued in the United States in 1972, and the last documented naturally occurring case of smallpox was recorded in Somalia in 1977. In 1980, the World Health Assembly declared smallpox eradicated (Henderson et al., 1999). It recommended that all routine smallpox vaccinations be suspended on a global scale and mandated that reference samples be retained only in two locations, the United States and the former USSR. All other smallpox stocks were to be destroyed.

Despite its supposed limited availability, smallpox, and other viruses, such as monkeypox, are potential biological weapons of mass destruction. Currently, vaccination is the only preventive and early prophylactic measure available for these diseases.

Vaccination is effective for pre- and post-exposure prophylaxis against smallpox. For post-exposure prophylaxis, though, the vaccine must be administered within four days of exposure (Henderson et al., 1999). Additionally, post-vaccination complications have been reported in 14 to 52 people per million, and one or two of these people may die from the vaccine. Those with deficient immune systems appear to be especially at risk. Complications include eczema vaccinatum, generalized vaccinia, ocular vaccinia, and progressive vaccinia. Complications of vaccination may be treated by administering vaccinia immune globin (VIG) obtained from vaccinated people (Henderson et al., 1999), although this reagent is in short supply and may present safety concerns, such as contamination with other viruses.

Current solutions to the problems that prevention and prophylaxis of viral biological weapons present could include: (1) the production of large stockpiles of current vaccine and VIG stocks; (2) the development of less virulent new vaccines; (3) the development of rapid diagnostic tools for smallpox; and (4) rapid screening and testing of licensed drugs.

Cidofovir, a viral DNA polymerase inhibitor licensed for the treatment of cytomegalovirus infections in AIDS patients, is being tested for use in the treatment of smallpox and post-vaccination complications (Smee and Sidwell, 2003). It has been shown to protect mice against lethal cowpox challenge (Martinez et al., 2000).

While the efficacy of intravenous administration of compounds against poxviral infections is based on mouse and primate studies, Cidofovir has some limitations for biodefense use. First, it is currently available in only intravenous injection form and oral administration has proven difficult. Thus, rapid self-administration of large civilian and military populations may not be possible. Second, severe side effects, including kidney damage, have been reported. Third, Cidofovir failed to completely protect immune deficient animals against lethal poxvirus infection in mice. Finally, Cidofovir-resistant poxviruses have been isolated in the laboratory. Taken together, these complications suggest that there is still a need for alternative drugs to meet the challenge of defending against viral biological weapons, including smallpox and monkeypox.

IFN-γ Human interferon-γ (IFN-γ) (available as ACTIMMUNE®, from InterMune, Brisbane, Calif.) is currently used to reduce the frequency and severity of serious infections associated with chronic granulomatous disease, a genetic disorder of the immune system that makes patients vulnerable to bacterial and fungal infections (Sechier et al., 1988).

INF-γ is a potent antiviral and immunomodulatory cytokine produced primarily by NK and T cells. IFN-γ has been demonstrated to be effective against experimental infections of several viruses including MCMV, RVFV, and poxviruses (Fennie et al., 1988; Morrill et al, 1991; Liu et al., 2004). Mice lacking IFN-γ receptors have been shown to be susceptible to poxvirus infection (Huang et al., 1993). In addition, depletion of IFN-γ with antibodies has been shown to allow more rapid virus dissemination (Karupiah et al., 1993), and recombinant vaccinia viruses expressing IFN-γ are greatly attenuated in immunodefficient mice (Kohonen-Corish et al., 1990). Finally, IFN-γ is capable of inducing nitric oxide (NO)-mediated inhibition of vaccinia virus by macrophages (Karupiah and Harris, 1995).

Other cytokines, IFN-α/β enhance cytotoxicity of NK cells and plays an important role in triggering dendritic cell-mediated acquired immunity (Biron, 1998).

Exogenous IFN-α/β and IFN-γ play an important role in antiviral defense in the following ways (Biron, 1998; Kontsek and Kontsekova, 1997; Billiau and Vandenbroeck, 2001): (1) by inducing an antiviral state through dsRNA-dependent protein kinase (PKR) and 2′, 5′-oligoadenylate synthetase (OAS)/RNAse L pathways; (2) by enhancing the CD8⁺ T cell-mediated cytolysis of infected cells by up-regulating the expression of MHC I molecules; and (3) by augmenting the migration of early response inflammatory leukocytes to the site of viral infection.

When an infection occurs, the body has an initial, rapid response to it. Before specific antibodies are produced, a branch of the immune system called the “innate immune system” is activated to produce the first proinflammatory reaction. In addition, the innate system instructs the other branch of the immune system, the “adaptive system,” to begin to produce the specific responses of T-cells and B-cells and the production of antibodies. Control of the innate immune system offers many opportunities for intercepting infections in their early stages and preventing some of the most severe effects, such as septic shock.

One aspect of the innate immune system that provides protection against viruses is the action of the antiviral cytokines. IFN-α/β protect neighboring cells from being infected by the same or unrelated viruses (Biron, 1997; Konstek and Konstekova, 1997; Van den Broek, et al., 1995). Mice lacking receptors for IFN-α/β and/or for IFN-γ demonstrate reduced resistance to poxvirus infections (Biron, 1998).

In addition to their direct antiviral activities, cytokines display immunomodulatory functions (Biron, 1997; Xing, 2000). For instance, IFN-α/β, IFN-γ, IL-2, and IL-12 are potent activators of macrophages and NK cells (Karupiah et al., 1991; Ramshaw et al., 1997). Vaccination of monkeys with simultaneous administration of IFN-α/β or the IFN inducer poly(I):poly(C) induced immunity that prevented viremia and alleviated local inflammatory reactions caused by vaccination (Bektemirov, 1980).

NO is a short-lived and short-distance radical gas that can diffuse easily in the absence of specific cellular receptors and provides macrophages with cytolytic or cytotoxic activity against microbes during infection and inflammation (MacMicking et al., 1997). NO functionally inhibits enzymes requiring iron and sulfur prosthetic groups by forming nitrosyl-iron-sulfur complexes (Harris et al., 1995), and may inhibit ribonucleotide reductase, an enzyme required for viral DNA synthesis (Lepivre et al., 1991; Kwon et al., 1991). The latter has been proposed as a mechanism to explain NO-mediated inhibition of vaccinia virus (VV) replication (Karupiah and Harris, 1995; Melkova and Esteban, 1994). NO is produced by inducible NO synthase (iNOS), which catalyzes the conversion of L-arginine to NO and L-citrulline. iNOS gene expression in macrophages is regulated by cytokines and microbial products via transcriptional induction (MacMicking, et al., 1997). Elevated iNOS gene expression has been associated with increases in NO production (Reiss and Komatsu, 1998). Activation of macrophages with IFN-γ and/or LPS results in increased iNOS expression and inhibition of replications of poxviruses, herpesviruses, retroviruses, and flaviviruses (Harris et al., 1995; Akarid et al., 1995; Croen, 1993; Lin et al., 1997). NO release has been shown to be inhibited in the presence of anti-CD14 antibody in PGN activated mice (Pugin et al., 1994), as neutralization of CD14 with its antibody blocks NO induction (Pugin et al., 1994).

It has previously been shown that NO-mediated inhibition of viral replication is neither host cell nor virus specific (Harris et al., 1995), and that IFN-γ synergizes with IFN-α/β and TNF-α in NO induction. For mouse peritoneal macrophages, IFN-γ is the only agent reported to date that has been shown to effectively induce NO when tested alone (Ding et al., 1998). A recombinant vaccinia virus expressing iNOS has been found to be attenuated in vivo (Rolph et al., 1996a). It has been speculated that NO induction in macrophages is one of the important antiviral strategies in infectious loci, where neutralizing antibodies are unavailable and macrophages are able to ingest and destroy the immature virus particles, thus preventing virus dissemination (Harris et al., 1995). However, NO production may contribute to the control of vaccinia virus (VV) growth, but other antiviral mechanisms, in the absence of NO, are able to mediate virus clearance. This hypothesis is based on the observation that treatment of VV-infected mice with an iNOS inhibitor does not affect the course of infection (Rolph et al., 1996a; Rolph et al., 1996b). It remains unclear whether the NO cytotoxic effector molecule plays an important role in human macrophage antimicrobial or antitumor activities, as it is difficult to show that human monocytes produce NO following cytokine activation.

Ribavirin Ribavirin, (1-β-D-ribofuranosyl-1, 2-4-triazole-3-carboxamide) (available as COPEGUS@, Hoffmann LaRoche, Inc., Nutley, N.J.), is a broad-spectrum antiviral drug, used for the treatment of RSV and HCV infections (Sidwell, 1980), which has shown certain beneficial effects on progressive vaccinia. It is effective against a least a dozen DNA viruses and 40 RNA viruses (Markland et al., 2000). Ribavirin has been approved as an inhalable antiviral agent for the treatment of RSV infections and can be taken orally, in combination with IFN-α, for the treatment of chronic hepatitis C (HCV) infections (Smee and Sidwell, 2003). Although ribavirin cannot fight HCV on its own, studies have shown that it does help alpha interferon work better. Ribavirin has been shown to suppress W induced tail lesions in a mouse model (DeClerq et al., 1976) and to effectively treat vaccinia keratitis in rabbits (Sidwell et al., 1973). In addition, ribavirin plus anti-vaccinia globulin provided benefit to an immunosupressed patient infected with vaccinia virus (Kesson et al., 1997). Ribavirin also demonstrates synergy with VIG and IFN-α (Smee and Sidwell, 2003). Ribavirin has been shown to protect mice against a lethal infection of a moderate dose of cowpox virus (Smee et al., 2000).

Ribavirin is an inhibitor of IMP dehydrogenase, an enzyme that converts IMP to XMP. This step is crucial for the biosynthesis of GTP and dGTP, thereby inhibiting viral DNA and RNA synthesis (DeClerq, 2001). Ribavirin also inhibits the function of transcriptase or replicase of reovirus (Rankin et al., 1989) as well as the formation of 5′-mRNA cap in W (Goswami et al., 1979). The main side effects of ribavirin include anemia during prolonged treatment in HCV patients (Rizetto, 1999).

Combinations of antiviral agents with different modes of action are likely to generate more potent antiviral efficacy. Antiviral synergy between IFN-α and ribavirin, between IFN-α/β and IFN-γ, and between IFN-γ and ribavirin has been demonstrated in the studies of the hepatitis C virus (HCV) (Moradpour and Blum, 1999), herpes simplex virus 1 (HSV-1) (Sainz et al., 2002), and SARS cronavirus (Sainz et al., 2004).

There is a need in the art for new treatments for viral infections, in particular those that can be used in biological warfare.

### BRIEF SUMMARY OF THE INVENTION

The invention aids in fulfilling this need by providing treatments and prophylaxis for viral infections comprising the compounds IFN-α, IFN-β, and IFN-γ, either alone or in combination with each other or with ribavirin. The treatments and prophylaxis of the invention allow for lowered dosages of IFN, reduced pro-inflammatory responses, and delayed the initiation time and reduced frequency of the IFN treatment required for the optimal protection.

### BRIEF DESCRIPTION OF THE DRAWINGS

FIG. 1 depicts the effect of doses and routes of administration of ribavirin on survival of vaccinia virus infected mice. Mice were infected with 50 LD₅₀ of virus, then treated with the indicated amounts of ribavirin or PBS (placebo), either intranasally (i.n.) or subcutaneously (s.c.).

FIG. 2 depicts the effect of intranasal administration of IFN-α and IFN-γ, at 5000 U per mouse, as a pre-exposure prophylaxis, on survival of vaccinia infected mice (8 LD50). The IFN administration started one day before the infection.

FIG. 3 depicts the effect of doses of IFN-γ on survival of vaccinia virus infected mice. Mice were infected with 50 LD₅₀ of virus, then treated with the indicated amounts of IFN-γ (U/mouse), or PBS (placebo), as indicated.

FIG. 4 depicts survival of vaccinia virus infected mice following intranasal administration of IFN-γ and ribavirin. Groups of mice were infected with 50 LD₅₀ of virus, then treated daily (i.n.) with IFN-γ (10,000 U/mouse/day), ribavirin (50 mg/kg), IFN-γ (10,000 U/mouse/day) and ribavirin (50 or 25 mg/kg), or PBS (placebo), as indicated.

FIG. 5 depicts the survival of vaccinia virus infected mice following intranasal administration of interferon gamma and ribavirin. Mice were infected with 8 LD50 of vaccinia virus, then treated with IFN-γ (2,000 U/mouse/day), ribavirin (25 and 100 mg/kg), IFN-γ (2,000 U/mouse/day) and Ribavirin (25 mg/kg), or PBS (placebo), as indicated.

### DETAILED DESCRIPTION OF THE PREFERRED EMBODIMENTS

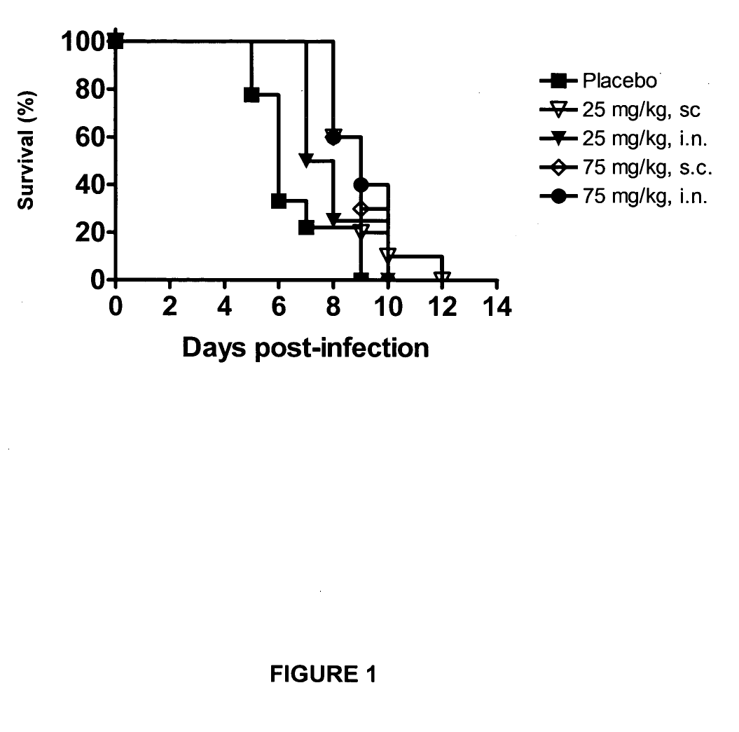

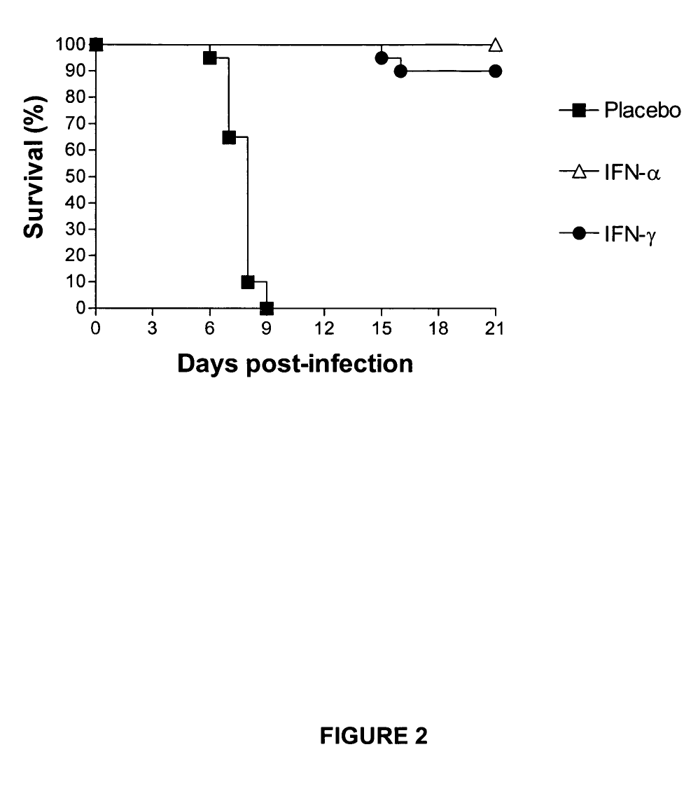

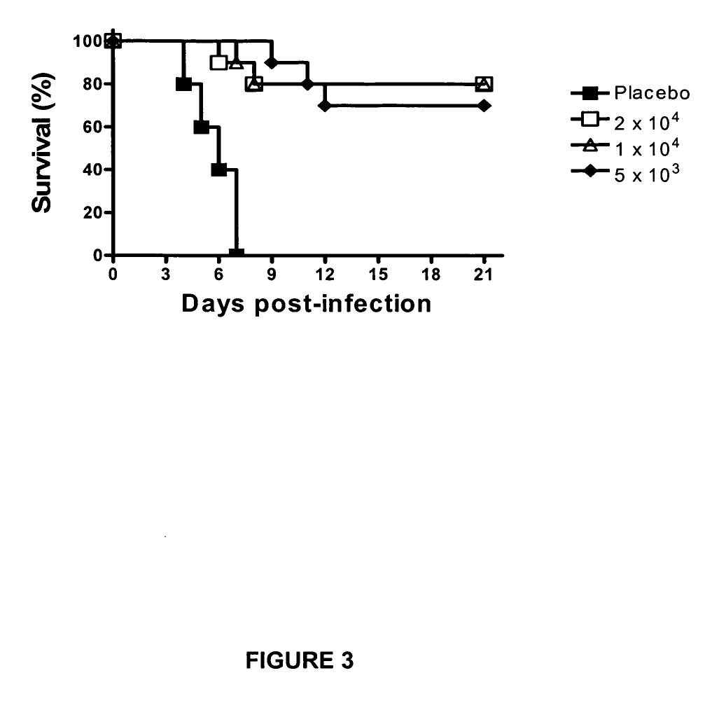

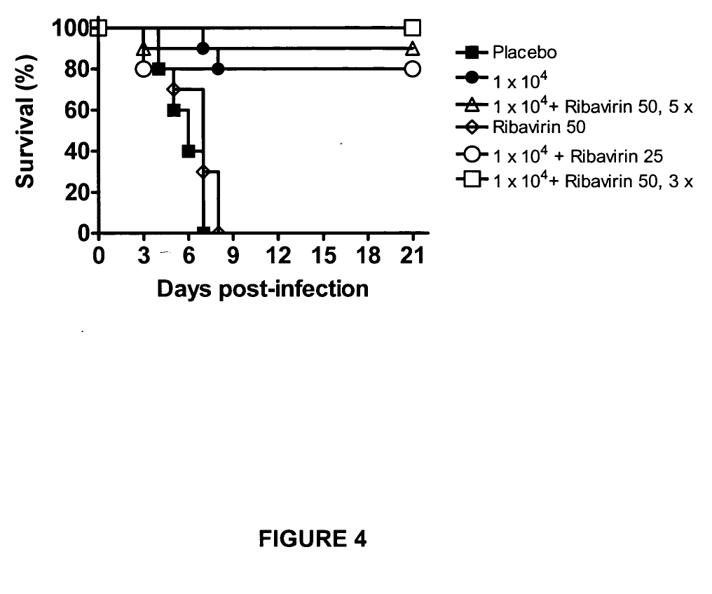

Like the protective effect of ribavirin against lethal infection by a moderate dose of cowpox virus in mice (Smee et al., 2000), ribavirin has now been shown to delay the death of mice infected with vaccinia (FIG. 1). Intranasal administration of IFN-α (5,000 U) and IFN-γ (5,000 to 20,000 U) have also been shown to lead to 70-80% protection against severe vaccinia infection (50 LD₅₀) in mice (FIGS. 2 and 3). Finally, this invention demonstrates that when ribavirin is included in IFN-γ treatment, there is a slight increase in the survival of vaccinia-infected mice (FIG. 4). This demonstrates that IFN-γ, and the combination of IFN-γ and ribavirin, are effective anti-poxviral treatments for poxvirus infection.

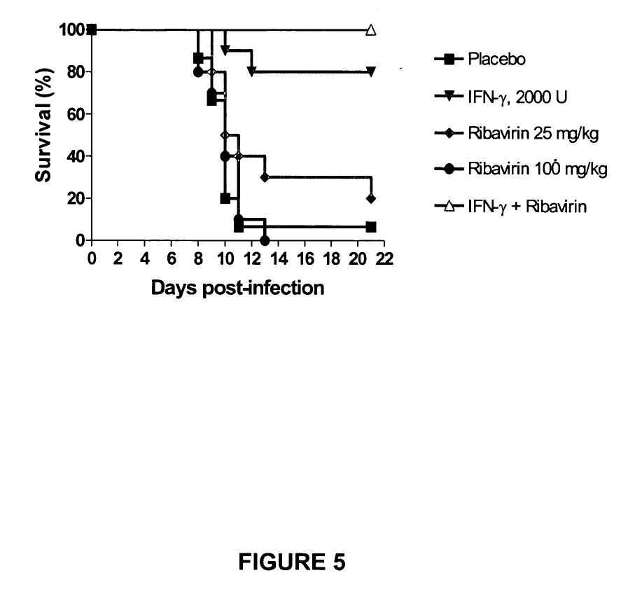

This invention includes the finding that mice infected with vaccinia and administered only ribavirin live 2-3 days longer than untreated mice (FIG. 1). This increase in survival time provides IFN-γ more time to exert its direct and indirect antiviral activities in a combined treatment. The combination of IFN-γ and ribavirin showed an increase in the survival of infected mice (FIGS. 4 and 5), although this increase was not statistically significant, possibly because of the relatively high dose of IFN-γ, which could have dominated the antiviral efficacy. Even though the increase in survival with the combination treatment was small, it is important because the initial increase in survival that ribavirin treatment alone provides will allow the delayed effects of IFN-γ to be unmasked and to sustain survival beyond the first 2-3 days.

The invention provides that combination treatments with ribavirin and IFN-γ reduce the IFN-γ-induced pro-inflammatory responses, such as those caused by cytokine (IL-6) and chemokine (MCP-1) production, which may contribute to the pathogenesis of infected hosts. Further studies with the mouse vaccinia model will demonstrate another feature of the invention, namely the synergy between IFN-γ and ribavirin, as well as the effects of such treatment on the host immune responses in infected mice.

In an embodiment of the invention, a method of treating a patient infected with a virus is provided. The method comprises: (A) administering IFN-α, IFN-γ, IFN-α plus IFN-γ, or ribavirin plus either IFN-α or IFN-γ to the patient; and (B) alleviating the effects of the virus in the patient.

In embodiments of the invention, the effects of the virus include, but are not limited to, death, viral load, fever, pneumonia, edema, malaise, headache, backache, skin lesions, mucous membrane lesions, bleeding, and dehydration.

In an embodiment of the invention, ribavirin and IFN are administered in admixture or sequentially to the patient. In one embodiment, ribavirin is administered before IFN, while in another embodiment, IFN is administered before ribavirin.

In embodiments of the invention, INF-γ is administered to the patient at 150 μg by subcutaneous injections. This treatment can typically last for four weeks or longer. Recombinant INF-γ can also be administered to a patient at doses of 0.01 to 2.5 mg/m² by alternating intramuscular and intravenous bolus injections with a minimum intervening period of 72 h. As known by those skilled in the art, administration of cytokines by inhalation, as provided for in the invention, will require significantly lower doses than those recited here by injection. These lower doses can reduce any adverse side effects of these cytokines.

IFN-α has been licensed to treat HCV infections. IFN-α can be administered at up to 300 million IU/m² subcutaneously, without adverse reactions. IFN-α can typically be administered at 2-20 million IU/m², either intravenously, intramuscularly, or subcutaneously. Treatments with INF-α at these doses can last for four weeks to six months or a year.

In other embodiments of the invention, ribavirin is administered to a patient at 880-1200 mg in two divided doses. In these embodiments, ribavirin can be administered orally, in pill form, or, preferably, it can be inhaled liposomes.

In embodiments of the invention, IFN and ribavirin are administered by other methods, including, but not limited to, intramuscularly, subcutaneously, and intravenously. In a preferred embodiment of the invention, IFN is administered intranasally to allow for self-administration (nasal spray or inhaler delivered powder) of a large number of individuals, for example, during a biological weapon attack.

In embodiments of the invention, IFN or IFN plus ribavirin are administered, sequentially or in admixture, five times daily, intranasally, and optionally, subcutaneously as well. Treatments can be continued for 3 days to 5 days. In the treatment of an existing infection, these compounds can be given one day after exposure to the virus. IFN-γ is also partly effective when the treatment is started two days after infection. In other embodiments, intranasal administration of IFN-γ can be sufficient.

The invention can be used for the treatment of smallpox, monkeypox, and smallpox vaccination related complications. The invention is also useful for the treatment of other respiratory viral infections, including, but not limited to, the common cold, SARS, and respiratory syncytial virus (RSV) infection. The invention can also be used for the treatment of other poxvirus infections including, but not limited to, molluscum contagiosum virus (MCV), which is a world wide opportunistic infection among AIDS patients.

In yet other embodiments of the invention, IFN can be purified recombinant IFN, and can be administered in liposomes, or as micro-encapsulated or pegylated compounds. Other forms that allow for stabilization in the blood are also encompassed by this invention. In a preferred embodiment, IFN-γ can be inhaled in the form of liposomes to increase bioavailability and reduce side effects associated with high dosages. Liposomal IFN-α and IFN-γ have been reported to be more effective against lung cancer and multidrug-resistant TB than native IFNs. In addition, IFNs can be conjugated to polyethylene glycol.

In another embodiment of the invention, the IFN-α, IFN-γ, or IFN plus ribavirin, are administered to achieve prophylaxis. In this embodiment, the compounds are administered sequentially or in a mixture before infection, optionally at least once before infection.

In a further embodiment of the invention, the prophylactic and therapeutic treatments of the invention can be used for individuals at some risk or even high risk for adverse effects of the smallpox vaccine currently available, including but not limited to those with deficient immune systems or suffering from eczema.

In yet other embodiments of the invention, the IFN-α, IFN-γ, IFN-γ plus ribavirin, or IFN-α plus ribavirin can be provided in a pharmaceutical composition including a pharmaceutically acceptable carrier.

A combination of IFN-α and IFN-γ or ribavirin plus either of these IFNs, are the preferred embodiments for treatment of poxvirus, including smallpox, infections.

IFNs are preferably administered intranasally as a therapeutic composition. Other routes of administration include oral, subcutaneous, intramuscular, intravenous, or intraperitoneal.

In one embodiment the composition of the invention can be used for pre-exposure prophylaxis. In another embodiment of the invention the composition is used for post-exposure treatment.

The treatments of the invention also encompass the use of IFN-β alone or in combination with ribavirin.

IFN-β can be used in doses ranging from 6MU of IFN-β, once daily intravenously, for six weeks up to 12 weeks or at 12 MU once daily subcutaneously for four weeks. Alternatively, IFN-β can be used in humans at a dose of 6 MU five times weekly for 24 weeks.

This invention will be described in greater detail in the following Examples.

### EXAMPLE 1

### Effect of Doses and Routes of Administration of Ribavirin on Survival of Vaccinia Virus Infected Mice

Groups of five BALB/c mice were treated daily (i.n.) with the indicated amounts of ribavirin via intranasal (i.n.) or subcutaneous (s.c.) route, or with PBS (placebo) for five consecutive days. All animals were infected with 50 LD₅₀ of VV on day 0 and were monitored 21 days for mortality. The treatments started one day after infection.

FIG. 1 demonstrates that, in addition to protecting mice against a lethal infection of a moderate dose of cowpox virus (Smee et al., 2000). Ribavirin delays the death of mice infected with vaccinia by 2-3 days.

### EXAMPLE 2

### Effect of Pre-exposure IFN Administration on Survival of Vaccinia Virus Infected Mice

Groups of 20 BALB/c mice were treated daily (i.n.) with 5×10³ U of IFN-α, IFN-γ, or PBS (placebo) for 5 consecutive days. All animals were infected with 8 LD₅₀ of W on day 0 and were monitored 21 days for mortality. The treatments started one day before infection.

FIG. 2 shows that intranasal administration of IFN-α and IFN-γ (each at 5×10³ U/mouse/day) resulted in 100% and 90% survival of vaccinia infected mice (8 LD₅₀).

### EXAMPLE 3

### The Effect of Doses of IFN-γ on Survival of Vaccinia Virus Infected Mice

Groups of 10 BALB/c mice were treated daily (i.n.) with indicated amounts of IFN-γ (U/mouse), or PBS (placebo) for five consecutive days. All animals were infected with 50 LD₅₀ of W on day 0 and were monitored for 21 days for mortality. The treatments started one day after infection.

FIG. 3 shows that intranasal administration of IFN-γ (5,000 to 20,000 U) led to 70-80% protection against severe vaccinia infection (50 LD50) in mice.

### EXAMPLE 4

### Survival of Vaccinia Virus Infected Mice Following Intranasal Administration of Interferon Gamma and Ribavirin

Groups of five to ten BALB/c mice were treated daily (i.n.) with IFN-γ (10,000 U/mouse/day), ribavirin (50 mg/kg), with IFN-γ (10,000 U/mouse/day) and ribavirin (50 or 25 mg/kg), or PBS (placebo) for five or three consecutive days. All animals were infected with 50 LD₅₀ of W on day 0 and were monitored for 21 days for mortality. The treatments were started one day after infection.

FIG. 4 shows that the addition of ribavirin in the IFN-γ treatment slightly increased the survival of vaccinia-infected mice.

### EXAMPLE 5

### Survival of Vaccinia Virus Infected Mice Following Intranasal Administration of Interferon Gamma and Ribavirin

Groups of ten BALB/c mice were treated daily (i.n.) with IFN-γ (2,000 U/mouse/day), ribavirin (25 and 100 mg/kg), IFN-γ (2,000 U/mouse/day) and Ribavirin (25 mg/kg), or PBS (placebo) for 5 consecutive days. All animals were infected with 8 LD₅₀ of vaccinia virus on day 0 and were monitored for 21 days for mortality. The treatments started one day after infection.

FIG. 5 shows that when a lower dose of virus (8 LD50) was used for infection, all animals that received 2,000 U of IFN-γ plus 25 mg/kg ribavirin survived, compared to a 80% survival of those received only IFN-γ (2,000 U) (FIG. 5), although this result was not statistically significant. In this experiment, treatment with a higher dose (5,000 U) of IFN-γ led a 100% protection.

### REFERENCES

The following references are cited herein. The entire disclosure of each reference is relied upon and incorporated by reference herein.

Akarid, K., Sinet, M., Desforges, B., and Gougerot-Pocidalo, M. A. (1995) Inhibitory effect of nitric oxide on the replication of a murine retrovirus in vitro and in vivo. J Virol. 69(11):7001-5.

Alvarez-Lafuente R, De Las Heras V, Bartolome M, Picazo J J, Arroyo R. (2004) Beta-interferon treatment reduces human herpesvirus-6 viral load in multiple sclerosis relapses but not in remission. Eur Neurol. 52(2):87-91. Epub 2004 Jul 22.

Billiau, A. and Vandenbroeck, K. (2001). IFNgamma. In: J. J. Oppenheim, M. Feldman, S. K. Durum, T. Hirano, J. Vilcek and N. A. Nicola (Eds), Cytokine reference: A compendium of cytokines and other mediators of host defense, pp. 641-688. Academic Press, New York.

Bektemirov, T. A, Burgasova, M. P., Kuznetsov, V. P, Rozina, E. E., and Andzhaparidze, O. G. (1980) Smallpox vaccination with protection by interferon and an interferon inducer. Vopr. Virusol: Smallpox vaccination with protection by interferon and an interferon inducer. Vopr Virusol. Jan-Feb;(1):76-8.

Biron, C. A. (1997) Activation and function of natural killer cell responses during viral infections. Curr Opin Immunol 1997. 9(1):24-34.

Biron, C. A. (1998) Role of early cytokines, including alpha and beta interferons (IFN-alpha/beta), in innate and adaptive immune responses to viral infections. Semin Immunol 10(5):383-90.

Cheng P N, Marcellin P, Bacon B, Farrell G, Parsons I, Wee T, Chang T T. (2004) Racial differences in responses to interferon-beta-1a in chronic hepatitis C unresponsive to interferon-alpha: a better response in Chinese patients. J Viral Hepat. 11:418-26.

Condos, R. Rom, W. N., and Schluger, N. W. (1997) Treatment of multidrug-resistance pulmonary tuberculosis with interferon-[gamma] via aerosol. Lancet, 349: 1513-15.

Croen, K. D. (1993) Evidence for antiviral effect of nitric oxide. Inhibition of herpes simplex virus type 1 replication. J Clin Invest. 91(6):2446-52.

Davis, G. L. (1999) Combination treatment with interferon and ribavirin for chronic hepatitis C. Clin. Liver Dis. 3(4): 811-26.

De Clercq, E., Luczak, M., Shugar, D., Torrence, P. F., Waters, J. A., and Witkop, B. (1976) Effect of cytosine, arabinoside, iododeoxyuridine, ethyldeoxyuridine, thiocyanatodeoxyuridine, and ribavirin on tail lesion formation in mice infected with vaccinia virus. Proc Soc Exp. Biol Med 151, 487-490.

De Clercq, E. (2001) Vaccinia virus inhibitors as a paradigm for the chemotherapy of poxvirus infections. Clin. Microbiol Rev 14, 382-397.

Ding, A. H., Nathan, C. F., and Stuehr, D. J. (1998) Release of reactive nitrogen intermediates and reactive oxygen intermediates from mouse peritoneal macrophages. Comparison of activating cytokines and evidence for independent production. J Immunol. 141(7):2407-12.

Fennie, E. H., Lie, Y. S., Low, M. A., Gribling, P., and Anderson, K. P. (1988) Reduced mortality in murine cytomegalovirus infected mice following prophylactic murine interferon-gamma treatment. Antiviral Res. 10, 27-39.

Goswami, B. B., Borek, E., Sharma, O. K., Fujitaki, J., and Smith, R. A. (1979) The broad spectrum antiviral agent ribavirin inhibits capping of mRNA. Biochem Biophys Res Commun 89, 830-836.

Halme, M. Massilta, P., Repo, H., Ristola, M., Tawskinen, E., Mattson, K., and Cantell., K. (1995) Inhaled recombinant interferon gamma in patients with lung cancer: Pharmacokinetics and effects on chemiluminescence responses of alverolar macrophages and peripheral blood neutrophils and monocytes. Int. J. Radiat. Oncol. Biol. Phys. 31: 93-101.

Harris, N., Buller, R. M., and Karupiah, G. (1995) Gamma interferon-induced, nitric oxide-mediated inhibition of vaccinia virus replication. J Virol. 69(2):910-15.

Henderson, D. A., Inglesby, T. V., Bartlett, J. G., Ascher, M. S., Eitzen, E., Jahrling, P. B., Hauer, J., Layton, M., McDade, J., Osterholm, M. T., O'Toole, T., Parker, G., Perl, T., Russell, P. K., and Tonat, K. (1999) Smallpox as a biological weapon: medical and public health management. Working Group on Civilian Biodefense. JAMA 281, 2127-2137.

Hensley L E, Fritz L E, Jahrling P B, Karp C L, Huggins J W, Geisbert T W. (2004) Interferon-beta 1a and SARS coronavirus replication. Emerg Infect Dis. 10(2):317-9.

Huang, S., Hendriks, W., Althage, A., Hemmi, S., Bluethmann, H., Kamijo, R., Vilcek, J., Zinkernagel, R. M., and Aguet, M. (1993) Immune response in mice that lack the interferon-gamma receptor. Science 259, 1742-1745.

Karupiah, G., Woodhams, C. E., Blanden, R. V., and Ramshaw, I. A. (1991) Immunobiology of infection with recombinant vaccinia virus encoding murine IL-2. Mechanisms of rapid viral clearance in immunocompetent mice. J Immunol. 147(12):4327-32.

Karupiah, G., Fredrickson, T. N., Holmes, K. L., Khairallah, L. H., and Buller, R. M. (1993) Importance of interferons in recovery from mousepox. J. Virol. 67, 4214-4226.

Karupiah, G., Harris, N. (1995) Inhibition of viral replication by nitric oxide and its reversal by ferrous sulfate and tricarboxylic acid cycle metabolites. J Exp. Med 181, 2171-2179.

Kesson, A. M., Ferguson, J. K., Rawlinson, W. D., and Cunningham, A. L. (1997) Progressive vaccinia treated with ribavirin and vaccinia immune globulin. Clin. Infect Dis 25, 911-914.

Kohonen-Corish, M. R., King, N. J., Woodhams, C. E., and Ramshaw, I. A. (1990) Immunodeficient mice recover from infection with vaccinia virus expressing interferon-gamma. Eur. J Immunol 20, 157-161.

Kontsek, P., and Kontsekova, E. (1997) Forty years of interferon. Acta Virol 41(6):349-53.

Kurzrock, R., Rosenblum, M. G., Sherwin, S. A., Rios, A., Talpaz, M., Quesada, J. R., and Gutterman, J. U. (1985) Pharmacokinetics, single-dose tolerance, and biological activity of recombinant gamma-interferon in cancer patients. Cancer Res. 45, 2866-2872.

Kwon, N. S., Stuehr, D. J., and Nathan, C. F. (1991) Inhibition of tumor cell ribonucleotide reductase by macrophage-derived nitric oxide. J Exp Med. 174(4):761-7.

Lepoivre, M., Fieschi, F., Coves, J., Thelander, L., and Fontecave, M. (1991) Inactivation of ribonucleotide reductase by nitric oxide. Biochem Biophys Res Commun. 179(1):442-8.

Lin, Y. L., Huang, Y. L., Ma, S. H., Yeh, C. T., Chiou, S. Y., Chen, L. K., and Liao, C. L. (1997) Inhibition of Japanese encephalitis virus infection by nitric oxide: antiviral effect of nitric oxide on RNA virus replication. J Virol. 71(7):5227-35.

Liu, G., Zhai, Q., Schaffner, D., Wu, A., Yohannes, A., Robinson, T., Maland, M., Wells, J., Voss, T., Bailey, C., and Aibek, K. (2004) Prevention of Lethal Respiratory Vaccinia Infections in Mice with Interferon and Interferon. FEMS Immunol Med Microbiol. 40: 201-206.

Lukaszewski, R. A., Brooks, T. J. (2000) Pegylated alpha interferon is an effective treatment for virulent Venezuelan equine encephalitis virus and has profound effects on the host immune response to infection. J Virol 74, 5006-5015.

MacMicking, J., Xie Q. W., and Nathan, C. (1997) Nitric oxide and macrophage function. Annu Rev Immunol. 15:323-50.

Markland, W.; McQuaid, T. J.; Jain, J.; Kwong, A. D. (2000) Broad-spectrum antiviral activity of the IMP dehydrogenase inhibitor VX-497: a comparison with ribavirin and demonstration of antiviral additivity with alpha interferon. Antimicrob. Agents Chemother. 44: 859-866.

Martinez, B. M., Smee, D. F., Kefauver, D., Thompson, E., and Huggins, J. W. (2000) Cidofovir protects mice against lethal aerosol or intranasal cowpox virus challenge. J Infect Dis 181(1):10-9.

Melkova, Z., and Esteban, M. (1994) Interferon-gamma severely inhibits DNA synthesis of vaccinia virus in a macrophage cell line. Virology. 198(2):731-5.

Moradpour, D., Blum, H. E. (1999) Current and evolving therapies for hepatitis C. Eur. J Gastroenterol. Hepatol. 11, 1199-1202.

Morgenstern B, Michaelis M, Baer P C, Doerr H W, Cinatl J Jr. (2005) Ribavirin and interferon-beta synergistically inhibit SARS-associated coronavirus replication in animal and human cell lines. Biochem Biophys Res Commun. 326(4):905-8.

Morrill, J. C., Czarniecki, C. W., and Peters, C. J. (1991) Recombinant human interferon-gamma modulates Rift Valley fever virus infection in the rhesus monkey. J Interferon Res 11, 297-304.

Munoz R, Castellano G, Fernandez I, Alvarez M V, Manzano M L, Marcos M S, Cuenca B, Solis-Herruzo J A. (2002) A pilot study of beta-interferon for treatment of patients with chronic hepatitis B who failed to respond to alpha-interferon. J Hepatol. 37:655-9.

Pugin, J., Heumann, I. D., Tomasz, A., Kravchenko, V. V., Akamatsu, Y., Nishijima, M., Glauser, M. P., Tobias, P. S., and Ulevitch, R. J. (1994). CD14 is a pattern recognition receptor. Immunity. 1, 509-516.

Ramshaw, I. A., Ramsay, A. J., Karupiah, G., Rolph, M. S., Mahalingam, S., and Ruby, J. C. (1997) Cytokines and immunity to viral infections. Immunol Rev 159:119-35.

Rankin, J. T., Jr., Eppes, S. B., Antczak, J. B., and Joklik, W. K. (1989) Studies on the mechanism of the antiviral activity of ribavirin against reovirus. Virology 168,147-158.

Reiss, C. S., and Komatsu, T. (1998) Does nitric oxide play a critical role in viral infections? J Virol. 72(6):4547-51.

Rizzetto, M. (1999) Therapy of chronic viral hepatitis: a critical view. Ital. J Gastroenterol. Hepatol. 31, 781-793.

Rolph, M. S., Cowden, W. B., Medveczky, C. J., and Ramshaw, I. A. (1996a) A recombinant vaccinia virus encoding inducible nitric oxide synthase is attenuated in vivo. J Virol. 70(11):7678-85.

Rolph, M. S., Ramshaw, I. A., Rockeft, K. A., Ruby, J., and Cowden, W. B. (1996b). Nitric oxide production is increased during murine vaccinia virus infection, but may not be essential for virus clearance. Virology 217, 470-477.

Sainz, B., Jr., Halford, W. P. (2002) Alpha/Beta interferon and gamma interferon synergize to inhibit the replication of herpes simplex virus type 1. J Virol 76, 11541-11550.

Sainz B Jr, Mossel E C, Peters C J, Garry R F. (2004). Interferon-beta and interferon-gamma synergistically inhibit the replication of severe acute respiratory syndrome-associated coronavirus (SARS-CoV). Virology. 10;329(1): 11-7.

Sechler, J. M., Malech, H. L., White, C. J., and Gallin, J. I. (1988) Recombinant human interferon-gamma reconstitutes defective phagocyte function in patients with chronic granulomatous disease of childhood. Proc. Natl. Acad. Sci. USA. 85, 4874-4878.

Shiratori Y, Nakata R, Shimizu N, Katada H, Hisamitsu S, Yasuda E, Matsumura M, Narita T, Kawada K, Omata M; IFN-beta Research Group. (2000) High viral eradication with a daily 12-week natural interferon-beta treatment regimen in chronic hepatitis C patients with low viral load. IFN-beta Research Group. Dig Dis Sci. 45:2414-21.

Sidwell, R. W., Allen, L. B., Khare, G. P., Huffman, J. H., Witkowski, J. T., Simon, L. N., and Robins, R. K. (1973) Effect of 1-beta-D-ribofuranosyll-1,2,4-triazole-3-carboxamide (virazole, ICN 1229) on herpes and vaccinia keratitis and encephalitis in laboratory animals. Antimicrob. Agents Chemother. 3, 242-246.

Sidwell, R. W. (1980) Ribavirin: In vitro antiviral activity., In: Smith, R. A. and Kirkpatrick, W. (Eds), Ribavirin: A Broad Spectrum Antiviral Agent. pp. 23-42. Academic Press, New York.

Smee, D. F., Bailey, K. W., and Sidwell, R. W. (2000) Treatment of cowpox virus respiratory infections in mice with ribavirin as a single agent or followed sequentially by cidofovir. Antivir. Chem Chemother. 11, 303-309.

Smee, D. F., Sidwell, R. W. (2003) A review of compounds exhibiting anti-orthopoxvirus activity in animal models. Antiviral Res 57, 41-52.

Uyama H, Enomoto H, Kishima Y, Yamamoto M, Yoshida K, Okuda Y, Hirotani T, Kuroda T, Ito H, Matsuda M, Terabayashi M, Noguchi S, Kawase I, Nakamura H. (2003) A pilot study of combination therapy with initial high-dose interferon and amantadine hydrochloride for patients with chronic hepatitis C with the genotype 1b virus. Hepatogastroenterology. 50:2112-6.

Van den Broek, M. F., Muller, U., Huang, S., Zinkernagel, R. M., and Aguet, M. (1995) Immune defence in mice lacking type I and/or type II interferon receptors. Immunol. Rev. 148:5-18.

Xing, Z. (2000) Current understanding of macrophage type 1 cytokine responses during intracellular infections. Histol Histopathol. 15(1):199-205.

What is claimed is:

1. A method of treating a patient infected with a virus, wherein the method comprises:
(A) administering IFN-α, IFN-γ, or IFN-γ plus ribavirin to the patient; and,
(B) alleviating the effects of the virus in the patient, wherein the virus is smallpox, monkey pox, respiratory syncytial virus (RSV) infection, molluscum contagiocum virus (MCV), or the virus that causes the common cold.

2. The method as claimed in claim 1, wherein the IFN-α, IFN-γ, or IFN-γ plus ribavirin, is or are administered before infection, as prophylaxis.

3. The method as claimed in claim 1, wherein the IFN-α, IFN-γ, or IFN-γ plus ribavirin, is or are administered after infection.

4. The method as claimed in claim 1, wherein the IFN-α, IFN-γ, or IFN-γ plus ribavirin, is or are administered intranasally.

5. The method as claimed in claim 1, wherein the IFN-α or IFN-γ is recombinant, liposome encapsulated, pegylated, or in native, isolated form.

6. The method as claimed in claim 1, wherein IFN-α, IFN-γ, or IFN-γ plus ribavirin, is or are combined with a pharmaceutical carrier.
